(19) United States
(12) Patent Application Publication
(10) Pub. No.: US 2005/0227267 A1
(43) Pub. Date: Oct. 13, 2005

(54) Method of identifying genetic regions associated with disease and predicting responsiveness to therapeutic agents

(75) Inventor: Joel S. Bader, Stamford, CT (US)

Correspondence Address: CURAGEN CORPORATION, 322 EAST MAIN STREET, BRANFORD, CT, 06405, US

(73) Assignee: CuraGen Corporation, New Haven, CT (US)

(21) Appl. No.: 11/051,167

(22) Filed: Feb. 4, 2005

Related U.S. Application Data
(63) Continuation of application No. 09/966,870, filed on Sep. 28, 2001.
(60) Provisional application No. 60/236,765, filed on Sep. 29, 2000.

(51) Int. Cl. C12Q 1/68; G06F 19/00; G01N 33/48; G01N 33/50

(52) U.S. Cl. 435/6; 702/20

(57) ABSTRACT

The invention relates to a method of identifying genetic regions related to disease and to predicting the response to therapeutic agents. The invention provides a method of identifying a genetic region associated with a disease and/or associated with responsiveness to a therapeutic agent.

### RELATED APPLICATIONS

This application is a continuation application of U.S. patent application Ser. No. 09/966,870, which claims priority to U.S. Ser. No. 60/236,765, filed Sep. 29, 2000. The contents of each application are incorporated herein by reference in their entirety.

### FIELD OF THE INVENTION

The present invention relates to a method of identifying genetic regions related to disease and to predicting the response to therapeutic agents.

### BACKGROUND OF THE INVENTION

Identifying genetic components underlying complex traits is an important goal of modern medicine. These traits include prevalent diseases, including cancer, metabolic disorders such as diabetes and obesity, cardiovascular disorders such as hypertension and stroke, and psychiatric disorders. Genetic complexity also underlies stratification of patient populations presenting a single disease phenotype into sub-classes whose disorders might have differing genetic components or different responses to particular therapeutics.

Studies that identify the underlying genetic variations that cause increased disease risk or affect drug response have typically depended on the availability of markers spaced throughout the genome. Although these types of studies have identified causative mutations for monogenic disorders, they have not been as successful in identifying genetic components for complex, polygenic traits.

More recently, single nucleotide polymorphisms (SNPs) have been suggested as an alternative marker set. These single nucleotide substitutions or deletions are typically biallelic variants and occur at sufficient density to permit whole-genome association studies in outbred populations, indicating that hundreds of thousands of individual SNPs will be required for a whole-genome scan.

In order to correct for multiple hypothesis testing, a significance level of 10⁻⁸ to 10⁻⁹ has been suggested, which implies a sample size requirement of several thousand individuals for adequate power to detect association. Although the costs involved in genotyping can be reduced by testing allele frequency differences between pools of DNA collected from individuals with extreme phenotypes, these tests are necessarily less powerful than individual genotyping and require even larger sample sizes.

Obtaining sample sizes sufficiently large for full-genome scans can be cumbersome and expensive. One approach for reducing the sample size requirements for pharmacogenomic studies is to focus on polymorphisms residing in a small set of candidate genes representing the drug target and the disease and drug response pathways. Sequencing a drug target gene in 100 individuals, for example, reveals polymorphisms present at a frequency of 2% or greater. These markers, usually SNPs, may then be used for association tests.

Haplotypes or diploid haplotype pairs constitute an alternative set of markers for an association test, and haplotype-based tests have been suggested for use in clinical studies. Nevertheless, haplotype-based tests require additional work relative to SNP-based tests, including direct sequencing or computational inference to identify haplotypes, and for now preclude less costly tests of pooled DNA. With the interest in haplotype-based tests growing, more guidance is needed by experimentalists weighing the relative merits of SNP-based and haplotype-based tests or choosing between tests based on haplotypes or haplotype pairs.

### SUMMARY OF THE INVENTION

The invention provides a method of associating a phenotype with the occurrence of a particular set of allelic markers that occur at a plurality of genetic loci in a population of individuals. The invention allows for association tests to be performed using reduced sample sizes.

The method includes identifying the form of the allelic marker occurring at a plurality of genetic loci in the nucleic acid of each individual of the population, wherein each genetic locus is characterized by having at least two allelic forms of a marker and wherein the phenotype is expressed by a trait that is quantitatively evaluated on a numeric scale. A set of the allelic markers present in the nucleic acid of each individual of the population is identified, and the numeric value corresponding to the phenotypic trait for each individual of the population is obtained. Next, a p-value based on a particular set of markers and the numeric value is determineded. The p-value provides the probability that the association of the phenotype with the particular set is due to a random association. A p-value less than a predetermined limit establishes the association of said phenotype with occurrence of a particular set of allelic markers that occur at a plurality of genetic loci in a population of individuals.

Any number of genetic loci can be examined using the methods of the invention. In some embodiments, the number of genetic loci is 2, 3, 4, 5 10, 15, 20, 25, 50 or 100 or more. The number of individuals examined in the methods of the invention can be, e.g., 50,000 or fewer; 25,000 or fewer; 10,000 or fewer; 5,000 or fewer; 1,000 or fewer; 500 or fewer, 200 or fewer, 100 or fewer; 50 or fewer; or 25 or fewer.

In some embodiments, at least one allelic marker is a single nucleotide polymorphism (SNP). Various combinations of the allelic markers of at least two genetic loci that are in linkage disequilibrium with each other constitute different haplotypes.

In some embodiments, the genetic locus is characterized by having two allelic forms of the marker.

In some embodiments, at least two genetic loci are in linkage disequlibrium with respect to each other. The loci can be in partial or complete linkage disequlibrium.

In some embodiments, at least two genetic loci include a set of super-SNPs.

The p-value can be obtained, e.g., using a regression analysis, analysis of variance, or a combination of these methods. In some embodiments the p-value is less than 0.1. For example the p-value can be less than 0.05, 0.03, 0.01 or 0.005.

In another aspect, the invention provides a method of estimating the number of individual samples required to establish the association of a phenotype with occurrence of a particular set of allelic markers that occur at a plurality of genetic loci in a population of individuals. The method includes determining the number of SNPs to be evaluated and combining consecutive SNPs that are in linkage disequilibrium into super-SNPs. The number of haplotypes is also determined, as is the estimated number of samples required.

In some embodiments, the number of SNPs plus the number of super-SNPs is smaller than the number of haplotypes, and estimating uses the formula provided on the last line of Table 1 in column 2 or column 3.

In some embodiments, the number of SNPs plus the number of super-SNPs is greater than the number of haplotypes, and estimating uses the formula provided on the last line of Table 1 in column 4.

In some embodiments, the number of haplotypes is 2 or 3, and estimating uses the formula provided on the last line of Table 1 in column 4 or column 5. In other embodiments, the number of haplotypes is 4 or more, and estimating uses the formula provided on the last line of Table 1 in column 5.

In a still further aspect, the invention provides a method for identifying a genetic region associated with a disease. The method includes providing a plurality of single-nucleotide polymorphisms and a plurality of haplotypes for one or more regions of a chromosome, and identifying the number of single-nucleotide polymorphisms of said plurality in at least weak linkage disequilibrium with each other on said chromosomal regions. The number of single-nucleotide polymorphisms in linkage disequilibrium is compared to the number of haplotypes in said chromosomal regions. A correlation test is then selected, wherein a single-nucleotide-based correlation test is selected if the number of single-nucleotide polymorphisms in linkage disequilibrium is smaller than the number of haplotypes and a number of haplotype-based correlation test is selected if the number of single-nucleotide polymorphisms in linkage disequilibrium is greater than the number of haplotypes.

In some embodiments, the haplotype-based correlation test is a regression test. In other embodiments, the haplotype-based correlation test is ANOVA test.

In another aspect, the invention provides a method for identifying a genetic region associated with responsiveness to an agent. The method includes providing a plurality of single-nucleotide polymorphisms and a plurality of haplotypes for one or more regions of a chromosome and identifying the number of single-nucleotide polymorphisms of said plurality in at least weak linkage disequilibrium with each other on said chromosomal regions. The number of single-nucleotide polymorphisms in linkage disequilibrium is compared to the number of haplotypes in said chromosomal regions; and a correlation test is selected. A single nucleotide-based correlation test is selected if the number of single-nucleotide polymorphisms in linkage disequilibrium is smaller than the number of haplotypes, thereby identifying a genetic region associated with responsiveness to an agent.

In some embodiments, the haplotype-based correlation test is a regression test. In other embodiments, the haplotype-based correlation test is ANOVA test.

The invention provides efficient and cost-effective association tests based on SNPs and hapolotypes. Also provided by the invention are methods of association employing quantitative traits characteristic of disease risk or clinical response using SNP-based and haplotype-based tests. A further advantage of the invention is that allows for association tests to be performed using reduced sample sizes.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graphic representation showing the expected significance levels for tests of 150 individuals, corrected for multiple hypothesis testing, are shown for a haplotype-based ANOVA test (thin dot-dash) and for haplotype-based (thick dot-dash), SNP-based (dash), and super-SNP-based (solid) regression tests. Smaller p-values are more significant. In the model, G=10 SNPs contribute a cumulative 5% to the total variance of a quantitative phenotype. The abscissa of the top panel, G/Γ, represents the extent of linkage disequilibrium as measured by consecutive correlated SNPs, and is related to the number of haplotypes H by Γ=log₂ H.

FIG. 2 is a graphic representation showing the sample size N required for a Type I error rate of 5%, corrected for multiple hypothesis testing, and 80% power to reject the null hypothesis, is shown for a haplotype-based ANOVA test (thin dot-dash) and for haplotype-based (thick dot-dash), SNP-based (dash), and super-SNP-based (solid) regression tests. In the model, G=10 SNPs contribute a cumulative 5% to the total variance of a quantitative phenotype. The abscissa of the top panel, G/Γ, represents the extent of linkage disequilibrium as measured by consecutive correlated SNPs, and is related to the number of haplotypes H by Γ=log₂ H.

FIGS. 3A-3F. is a graphic representation showing comparisons between SNP-based and haplotype-based tests, the total number of SNPs is fixed at 20. The number of causative SNPs is 1 (left panels, 3A and 3D), 3 (middle panels, 3B and 3E), or 10 (right panels, 3C and 3F). The number of haplotypes, H, is varied from 1 to 100 within each panel. The additivevariance per SNP is fixed at 0.025. The top series of panels illustratesthe expected significance for a fixed population size of 300, and the bottomseries illustrates the population size required to attain a p-value of 0.05(5% false-positive rate including the multiple-testing correction) and a power of 0.8 (20% false-negative rate), for the haplotype-pair ANOVA test (dot-dashed line), the haplotype regression test (dashed line), and the SNP regression test (solid line). Haplotype-based tests and SNP-based tests cross in power when the number of haplotypes is just larger than the number of causative SNPs.

FIGS. 4A-4F. Same as FIG. 3, except the total the total additive variance is fixed at 0.075, implying an additive variance per SNP that varies from 0.075 (1 causative SNP) to 0.0075 (10 causative SNPs). The number of causative SNPs is 1 (left panels, 4A and 4D), 3 (middle panels, 4B and 4E), or 10 (right panels, 4C and 4F). The number of haplotypes, H, is varied from 1 to 100 within each panel. Haplotype-based tests and SNP-based tests cross in power when the number of haplotypes is just larger than the number of causative SNPs.

### DETAILED DESCRIPTION OF THE INVENTION

The present invention provides methods for associating phenotypes with particular sets of allelic markders. The methods are based in part on an analysis of the relative power of association tests based on SNPs and haplotypes. The methods are particularly sutiable for identying quantitative traits characteristic of disease risk or clinical response. The methods described herein provide for simple, analytical estimates of the relative efficiency of SNP-based and haplotype-based tests.

The present invention discloses the power of association studies using regression tests and ANOVA to identify SNP-based and haplotype-based markers for quantitative traits. Results derived from analytic theory based on an underlying variance components model indicate that ANOVA tests of haplotype pairs should only be used when the number of haplotypes is small. When the number of haplotypes increases beyond 4 or 5, a haplotype-based regression test has greater power. When the extent of linkage disequilibrium is difficult to establish, haplotype-based tests are more powerful than SNP-based tests if the number of haplotypes is less than the number of SNPs, while SNP-based tests are more powerful if there are fewer SNPs than haplotypes. The latter condition almost certainly holds when large genomic regions are tested for association. When the extent of linkage disequilibrium is evident because of correlations between individual SNPs, regression tests performed using super-SNPs, blocks of correlated SNPs, have the greatest power.

Simple formulas are provided for the experimentalist to estimate sample size requirements and p-values under each of these tests. It is shown in the Examples that these predictions agree with literature comparisons between SNP-based and haplotype-based tests, including findings that tests based on multi-locus markers, here termed super-SNPs, can have greater power than tests based on SNPs alone. The invention also provides that increasing the sample size of a study is more important than increasing the number of SNPs once the density of SNPs is comparable to the length scale of linkage disequilibrium.

While stronger linkage disequilibrium between SNPs implies fewer haplotypes, a small number of haplotypes does not necessarily imply strong linkage. A better estimate of the extent of linkage disequilibrium may be the typical number of consecutive SNPs correlated between different haplotypes, as demonstrated in Example 2.

Overall, the invention provides a simple set of guidelines for designing an association test for a candidate gene or drug target. First, identify the SNPs or haplotypes for one or more candidate genes. Consecutive SNPs found to be in linkage disequilibrium should be combined into a single super-SNP. When the number of SNPs and super-SNPs is smaller than the number of haplotypes, the SNP-based regression test is more powerful and should be used to calculate the required sample sizes; otherwise, haplotype-based tests are more powerful. With two or three haplotypes, the ANOVA test and the regression test have similar power and may both be used to estimate sample size requirements. With four or more haplotypes, the regression test is more powerful and should be used instead of ANOVA.

SNP-Based Phenotype Models

A variance components model is used to describe the dependence of an individual's phenotype on its genotype (Falconer et al., Introduction to Quantitative Genetics. Prentice Hall, New York (1996)). This quantitative model may also be applied to a haplotype relative risk model for disease susceptibility in which the risk from haplotypes are multiplicative and each risk factor is proportional to an exponential of an underlying quantitative trait (Terwilliger et al., Hum. Hered. 42: 337-346, 1992).

In the variance components model, the quantitative phenotype is denoted X and is standardized to have zero mean and unit variance. Several quantitative trait loci, here modeled as biallelic markers or SNPs, are assumed to contribute to the phenotypic value. Individual SNPs may occur within the same gene, and the total number of SNPs is G. The alleles for a particular SNP γ,γ=1 to G, are labeled A_{γ1} and A_{γ2}, with respective frequencies p_{γ} and 1−p_{γ1} in an unselected population. Hardy-Weinberg equilibrium is assumed separately for each SNP (but not for the joint distribution of SNPs γ and γ′), and the probabilities of the genotypes A_{γ1}A_{γ1}, A_{γ1}A_{γ2}, and A_{γ2} A_{γ2} are therefore p_{γ}², 2p_{γ}(1−p_{γ}), and (1−p_{γ})². The frequency of allele A_{γ1} for each individual is either 1, 0.5, or 0, and is denoted f_{γ}. The variance of f_{γ} is denoted σ_{f}_{_{γ}}², with
σ_{f}_{_{γ}}²=p_{γ}²·(1)+2p_{γ}(1−p_{γ})·(¼)+(1−p_{γ})²·(0)=p_{γ}(1−p_{γ})/2.

The effect of allele A_{γ1} is assumed to be purely additive with respect to allele frequency, a shift of a_{γ}/2 for each copy inherited. The shifts in phenotypic value are therefore a_{γ}−μ_{γ} for the A_{γ1} A_{γ1} homozygote, −μ_{γ} for the heterozygote, and −a_{γ}−μ_{γ} for the A_{γ2} A_{γ2} homozygote, where the constant μ_{γ}=a_{γ}(2p_{γ}−1) ensures that X has zero mean. This SNP contributes a phenotypic variance of σ_{γ}²,
σ_{γ}²=2p_{γ}(1−p_{γ})a_{γ}²,
to the total phenotypic variance of 1. For a polygenic trait, the variance σ_{γ}² contributed by any individual SNP is small compared to the residual variance 1−σ_{γ}²≈1 from other genetic and environmental factors. The expected value of σ_{γ}² is defined as σ_{G}²,

$${σ}_{G}^{2} = {G}^{- 1} {\sum }_{γ = 1}^{G} {σ}_{γ}^{2} ,$$

the mean of the individual variances. The fractional variance explained by all the SNPs together, Gσ_{G}², may also be much smaller than 1. Note that if the effect of a particular SNP is not purely additive, an additive effect can nevertheless be constructed by defining a_{γ} as half the difference in phenotypic shift between A_{γ1} and A_{γ2} homozygotes minus d_{γ}(2p_{γ}−1), where d_{γ} is the difference between the phenotype shift for heterozygotes and the midpoint of the shifts for homozygotes. This approach is generally valid for alleles with dominant, recessive, or multiplicative effects; it fails only for very rare recessive alleles and, correspondingly, for very common dominant alleles. In these extreme cases, however, the additive variance vanishes and associations are difficult to detect without recourse to highly selected populations.
Haplotypes

The G individual SNPs may occur in up to 2^{G} distinct allelic combinations. Due to linkage disequilibrium, however, a smaller subset of H haplotypes are assumed to occur in a test population. Using η to label the haplotype, η=1 to H, the phenotypic shift for an individual with haplotypes η and η′ is defined in analogy to the SNP shifts as (a_{η}+a_{η′})/2 , where

$${a}_{η} = {\sum }_{γ = 1}^{G} [ P ( {A}_{γ1} \vert η ) - P ( {A}_{γ2} \vert η ) - ( 2 {p}_{γ} - 1 ) ] {a}_{γ} .$$

The term P(A_{γ1}|η) has value 1 if haplotype η has allele A_{γ1} and is 0 otherwise. Similarly, P(A_{γ2}|η)=1 if haplotype η has allele A_{γ2} and is 0 otherwise. The difference in these terms, either +1 or −1, less its mean value 2p_{γ}−1, multiplies a_{γ} to yield the phenotypic shift in haplotype η due to the phase of SNP γ and is summed over all G SNPs.

While the precise value of a_{η} depends on the particular alleles occurring in haplotype η, the distribution of values of a_{η} may be estimated by considering the term P(A_{γ1}|η)−P(A_{γ2}|η) to be a random variable taking the value +1 with probability p_{γ} and the value −1 with probability 1−p_{γ}. This mean probability approximation recovers the SNP allele frequencies p_{γ} and ensures that the mean of a_{η} is zero. The variance Var(a_{η}) may be obtained under a random phase approximation in which the directions of the shifts a_{γ} are uncorrelated. With this assumption, the variance of the sum over SNPs is the sum of the individual variances even if the SNP allele frequencies are correlated. The variance of a_{η} arising from SNP γ is
p_{γ}[1−(2p_{γ}−1)]²a_{γ}²+(1−p_{γ})[−1−(2p_{γ}−1)]²a_{γ}²=4p_{γ}(1−p_{γ})a_{γ}²=2σ_{γ}².
The final variance for the distribution of haplotype-dependent shifts a_{η} is
Var(a_{η})=2Gσ_{G}²,
where σ_{G}² is the mean SNP variance as previously defined.

The mean phenotypic shift contributed by haplotype η is p_{η}²a_{η}+2p_{η}(1−p_{η})(a_{η}/2), or simply p_{η}a_{η}. The phenotypic variance contributed by this haplotype is defined as σ_{η}²,
σ_{η}²=p_{η}²a_{η}²+2p_{η}(1−p_{η})(a_{η}/2)²−(p_{η}a_{η})²=(½)p_{η}(1−p_{η})a_{η}².
When the number of haplotypes is large, the probability p_{η} for each haplotype is small and σ_{η}²≈p_{η}a_{η}²/2. The mean value of σ_{η}² is defined as σ_{H}²,

$${σ}_{H}^{2} = {H}^{- 1} {\sum }_{η = 1}^{H} {σ}_{η}^{2} = {H}^{- 1} {\sum }_{η = 1}^{H} {p}_{η} {a}_{η}^{2} / 2 = ( G / H ) {σ}_{G}^{2} ,$$

where it is assumed that p_{η} and a_{η} are uncorrelated. Note that the total haplotype-based phenotypic variance, Hσ_{H}², equals the total SNP-based phenotypic variance, Gσ_{G}².

In the special case that only one of the SNPs has a non-zero phenotypic shift a_{γ}, each haplotype η will have a phenotypic shift a_{η} of either 2(1−p_{γ})a_{γ} or −2p_{γ}a_{γ}, depending on whether A_{γ1} or A_{γ2} is included. The corresponding values for σ_{η}² will be p_{η}(1−p_{η})σ_{γ}² multiplied by either p_{γ}/(1−p_{γ}) or (1−p_{γ}/p_{γ}). Assuming that A_{γ1} is the minor allele with p_{γ} much smaller than 1 and that the haplotype frequency p_{η} is also much smaller than 1,
σ_{η}²=(p_{η}/p_{γ})σ_{γ}²
is the result for the variance due to the haplotype. A reasonable assumption is that the ratio p_{η}/p_{γ} is close to (1/H)/(1/G), yielding σ_{η}²=(G/H)σ_{γ}² as before.
Super-SNPs

When the number of haplotypes H is significantly smaller than the number of SNPs G, linkage disequilibrium must exist between certain of the SNPs. The extent of linkage disequilibrium between a pair of SNPs γ and γ′ is traditionally expressed in terms of the factor ρ_{γγ′}²,
ρ_{γγ′}²=(p₁₁p₂₂−p₁₂p₂₁)²/[p_{γ}(1−p_{γ})p_{γ}(1−p_{γ′})],
where pᵢⱼ is the frequency with which alleles A_{γi} and A_{γ′j} appear in phase on the same chromosome and, as before, p_{γ} and p_{γ′} are the frequencies of the A_{γ1} and A_{γ′1} alleles. When the minor-allele frequencies of the two SNPs are identical, the factor ρ² ranges from 1 for complete linkage to 0 for no correlation.

When linkage disequilibrium exists, the additive variance measured for a SNP-based marker may includes contributions from other SNPs. The observed additive variance for a SNP γ, denoted σ_{γ}²(obs), is

$${σ}_{γ}^{2} ( obs ) = {\sum }_{{γ}^{′} = 1}^{G} {ρ}_{{γγ}^{′}}^{2} {σ}_{{γ}^{′}}^{2} ,$$

where the terms σ_{γ′}² are the underling SNP-based variance components and include the self-contribution σ_{γ}². This is the precise relationship used to analyze association tests of neutral markers in linkage disequilibrium with causative mutations Ott et al., Analysis of Human Genetic Linkage, Johns Hopkins University Press, Baltimore, 1999; Falconer et al., Introduction to Quantitative Genetics, Prentice Hall, New York, 1996) The expected value of σ_{γ}²(obs) is estimated by noting that H haplotypes correspond to complete equilibrium between an effective number of Γpolymorphisms such that 2^{Γ}=H, or Γ=log₂ H. This suggests that linkage disequilibrium between SNPs extends approximately G/ΓSNPs, beyond which SNPs are essentially uncorrelated. The extremes are weak linkage, G/Γ=1, and strong linkage, G/Γ=1.

A simple model spanning the regime from weak linkage to strong linkage is that the G SNPs exist in Γ blocks of G/ΓSNPs, with perfect correlation within blocks and no correlation between blocks. The perfectly-correlated blocks are termed super-SNPs, and each SNP within a super-SNP has an identical observed additive variance. The use of a similar type of structure, termed a trimmed haplotype, has been previously suggested in the context of linkage analysis (MacLean et al., Am. J. Hum. Genet. 66:1062-75, 2000). If sequence data are available, then the extent of linkage disequilbrium G/Γ may be related to the average number of SNPs over which two haplotypes remain in phase.

The expected variance for a super-SNP is termed σ_{Γ}², equal to the variance σ_{γ}²(obs) observed for any of its component correlated SNPs. Furthermore, because of the correlation within a super-SNP block,
σ_{Γ}²=(G/log₂ H)σ_{G}²,
where G/log₂ H is the number of SNPs within the block. Because the blocks are uncorrelated, the variance summed over super-SNPs is identical to the variance summed over SNPs or haplotypes,
Γσ_{Γ}²=Hσ_{H}².
Since Γ=log₂ H, Γ is smaller than H and the phenotype variance explained by a super-SNP is expected to be larger than that explained by a haplotype. Also, since the number of haplotypes H≦2^{G}, Γ is usually smaller than G and a typical super-SNPs explain more phenotypic variance than does a typical SNPs.
Extreme Phenotypic Variance

Association tests are most sensitive to markers, here SNPs, haplotypes, and super-SNPs, conferring the greatest variation to the phenotype. Here the expectations for these extreme values are related to the variance terms σ_{G}², σ_{H}², and σ_{Γ}² for the various markers.

Under the phenotype model, the set of phenotypic shifts for M markers, either G SNPs, H haplotypes, or Γ super-SNPs, is drawn from a normal distribution with variance denoted σ_{M}². The probability that the largest positive shift confers a variance smaller than an extreme value σₑₓ² is [Φ(σₑₓ/σ_{M})]^{M}, where Φ(z) is the cumulative standard normal distribution for normal deviate z (Weisstein, The CRC Concise Encyclopedia of Mathematics. CRC Press, Boca Raton (1999). The expected median for the extreme value is obtained by setting [Φ(σₑₓ/σ_{M})]^{M} to 0.5. The median grows very slowly with the number of markers. For 5 markers, the result is (σₑₓ/σ_{M})=1.13; for 10 markers, (σₑₓ/σ_{M})=1.50; and for 100 markers, (σₑₓ/σ_{M})=2.46. The slow growth may be derived from the asymptotic expansion of Φ(z) valid for large z (Mathews et al., Mathematical Methods of Physics, Second Edition. Benjamin/Cummings, London. (1970)).
Φ(z)≈1−(2πz²)^{-0.5}exp(−z²/2)≈exp[−(2πz²)^{-0.5}exp(−z²/2)].
The approximate implicit solution for σₑₓ is
(σₑₓ/σ_{M})²≈2 ln [M/(2π)^{0.5}z ln(2)] with only a logarithmic dependence on M.

The simplifying assumption is made that σₑₓ≈σ_{M} and use the root-mean-square variance as an estimate of the extreme value. A similar approximation for the most extreme positive shift a_{η} for a haplotype is the standard deviation of the distribution for a_{η}, or (2Hσ_{H}²)^{0.5}. The corresponding most extreme negative shift is −(2Hσ_{H}²)^{0.5}.

Regression Test for Association

A suitable test statistic for either association of a SNP-based or haplotype-based marker with a quantitative phenotype is the coefficient b₁ for a regression model of the phenotypic value on the marker dose ((Falconer et al., 1996; SNEDECOR et al., Statistical Methods, Eighth Edition. Iowa State University Press, Ames (1989))
Xᵢ=b₁δfᵢ+εᵢ.

The N individuals included in the sample are specified by the index i. The difference between the marker frequency in individual i and in the total sample is δfᵢ, and the residual εᵢ is uncorrelated with δfᵢ. The expected value for b₁ is
b₁=σ_{M}/σ_{f},
where σ_{M}² is the additive variance of the marker, either σ_{γ}²(obs) for a SNP-based test or σ_{η}² for a haplotype-based test, and σ_{f}² is the variance of the marker frequency and equals p(1−p)/2 for a marker under Hardy-Weinberg equilibrium with frequency p. Since the variance of εᵢ is close to 1 when σ_{M}² is small, the variance of the estimator for b₁, σ_{b}², is the same under the null hypothesis, b₁=0, and the alternative hypothesis, b₁>0, and
σ_{b}²=1/N σ_{f}²
for a one-sided test.

Combining the expected value for the regression coefficient with the standard deviation of the estimator, the expected p-value for a one-tailed test for a marker with additive variance σ_{M}, using a Bonferroni correction for M multiple tests, is
p-value=1−[Φ(N^{0.5}σ_{M})]^{M}. (1)
Using the asymptotic expansion for Φ(z) yields
p-value≈M (2πN σ_{M}²)^{-0.5}exp(−N σ_{M}²/2) as an approximation valid for small p-values.

For a corrected final Type I error rate of α, the uncorrected p-value for a significant finding must be smaller than α/M. The Type II error rate β has no multiple testing correction. Defining the normal deviates z_{α/M}=Φ⁻¹(1−α/M) and z_{1-β}=Φ⁻¹(β), the resulting sample size required to detect a marker contributing phenotypic variance σ_{M}² with power 1−β is
N_{REGR}=(z_{α/M}−z_{1-β})²/σ_{M}². (2)

A simplified approximation for the sample size may be obtained by noting that z_{α/M} is typically larger than z_{1-β}. When α=0.05, M=10, and 1−β=0.8, for example, z_{α/M} =2.58 while z_{1-β}=−0.84. Neglecting z_{1-β} relative to z_{α/M} (or setting the power to 50%) yields
N≈2 ln(M/α)/σ_{M}².
The logarithmic term arises from the asymptotic expansion z_{α}˜2 ln(1/α) valid for small α.
ANOVA Test for Haplotype Association

Analysis of variance (ANOVA) may also be used to test for association between haplotype pairs and a quantitative phenotype. In a typical ANOVA test, N individuals are sorted into K=H(H+1)/2 distinct haplotype pairs and the between-genotype phenotypic variance is compared to the within-genotype phenotypic variance. A significant finding in an ANOVA test is approximately equivalent to detecting a significant difference in mean phenotype value for at least one of the C=K(K−1)/2 possible pairwise comparisons. The most significant finding will typically arise from the difference Δ in mean phenotypic value between the pair of genotypes with the most extreme positive and negative shifts.

The expected maximum difference Δ is obtained from the distribution of a_{η} as Δ=2[Var(a_{H})]^{0.5}, or (8Hσ_{H}²)^{0.5}. The variance for this test statistic is
σ²=σ_{R}²[(1/n)+(1/n′)],
where n and n′ are the number of individuals in the total sample size of N in the two extreme classes. Under the mean probability approximation, each p_{η} is 1/H. If the most extreme phenotypic shifts correspond to homozygous genotypes, then n and n′ are both approximately N/H² and the variance is σ²=2H²/N. If the genotypes with extreme phenotype values are both heterozygous, the variance is H²/N. The additive model suggests that homozygotes will be at least tied for the maximum phenotypic shift. The p-value for the comparison of extreme phenotypes is
p-value=1−[Φ(Δ/σ)]^{C}=1−[Φ(2σ_{H} N^{0.5}J^{0.5}/H^{0.5})]^{C}, (3)
where the factor of C is the correction for multiple hypothesis testing and J=1 if homozygotes are extreme, 2 if heterozygotes are extreme, and 1.5 if one homozygote and one heterozygote are extreme.

As with the regression test, the residual variance σ_{R}² is close to 1, and an expression yielding the required sample size is 1/σ²=(z_{α/C}−z_{1-β})²/Δ², or
N_{ANOVA}=(z_{α/C}−z_{1-β})²H/4Jσ_{H}². (4)
The ratio N_{ANOVA}/N_{REGR} of the sample size required for an ANOVA test, relative to that required for a series of H regression tests, is obtained from the ratio of Eq. 4 to Eq. 2. An estimate for this ratio, valid when z_{α/C} and z_{α/H} are both large compared to z_{1-β}, is
N_{ANOVA}/N_{REGR}≈(H/4J)ln(C/α)ln(H/α).
The logarithmic dependence varies slowly, and the factor H/4J explains most of the relative efficiency. When the number of haplotypes is small, ANOVA is more powerful. A cross-over occurs near H=4 if homozygotes are extreme and near H=8 if heterozygotes are extreme. Beyond the cross-over, the regression test is more powerful.
Comparison of Tests using SNPs, Haplotypes, and Super-SNPs

The significance levels expected for an association test and the sample level required to attain a pre-specified significance threshold are compared for statistical tests based on SNPs, haplotypes, and super-SNPs. The regression test is applied to all three, and the haplotype-based ANOVA test assuming homozygotes are most extreme is analyzed as well. A summary of the equations used for this analysis is provided in Table I.

TABLE I
Summary of association tests

| | | | | |
|---|---|---|---|---|
| Marker type | SNP | Super-SNP | Haplotype | Haplotype |
| Test | Regression | Regression | Regression | ANOVA |
| Number of | G | Γ ≈ log₂H or | H | H |
| markers | | G/(# of | | |
| | | consecutive | | |
| | | correlated | | |
| | | SNPs) | | |
| Phenotypic | Gσ_{G}² | Γσ_{Γ}² | Hσ_{H}² | Hσ_{H}² |
| variance | | | | |
| explained by | | | | |
| markers | | | | |
| Observed | σ_{G}² (weak | σ_{Γ}² = (G/Γ)σ_{G}² | σ_{H}² = (G/H)σ_{G}² | σ_{H}² |
| variance per | linkage) or | | | |
| marker | σ_{Γ}² (strong | | | |
| | linkage) | | | |
| p-value for N | 1 − [Φ(N^{0.5} σ_{G})]^{G} | 1 − [Φ(N^{0.5} σ_{Γ})]^{Γ} | 1 − [Φ(N^{0.5} σ_{H})]^{H} | 1 − {Φ[2(NJ/H)^{0.5} σ_{H}]}^{C} |
| individuals | (weak linkage) or | | | with J = 1, 1.5 or 2; |
| | 1 − [Φ(N^{0.5} σ_{Γ})]^{G} | | | C = K(K − 1)/2; and |
| | (strong linkage) | | | K ≈ H(H + 1)/2 |
| N for Type I | (z_{α/G} − z_{1-β})²/σ_{G}² | (z_{α/Γ} − z_{1-β})²/σ_{Γ}² | (z_{α/M} − z_{1-β})²/σ_{H}² | (z_{α/C} − z_{1-β})² H/4Jσ_{H}² |
| error α and | (weak linkage) or | | | |
| power 1 − β | (z_{α/G} − z_{1-β})²/σ_{Γ}² | | | |
| | (strong linkage) | | | |

The number of SNPs, G, is set to 10 for these examples, and the fraction of the total phenotypic variance explained by these 10 SNPs, Gσ_{G}², is 5%. This relatively large value reflects a model in which SNPs in a known drug target are tested for association with drug response. The number of haplotypes, H, is varied from a maximum of 1024, no linkage between SNPs, to a minimum of 2, complete linkage disequilibrium. The number of super-SNPs, Γ, is log₂ H, and the extent of linkage disequilibrium measured in SNPs, G/Γ, varies from 1 (no linkage) to 10 (complete disequilibrium). The mean phenotypic variance contributed per haplotype, σ_{H}², is (G/H)σ_{G}², and the observed variance per SNP and the mean variance per super-SNP are both σ_{Γ}²=(G/Γ)σ_{G}².

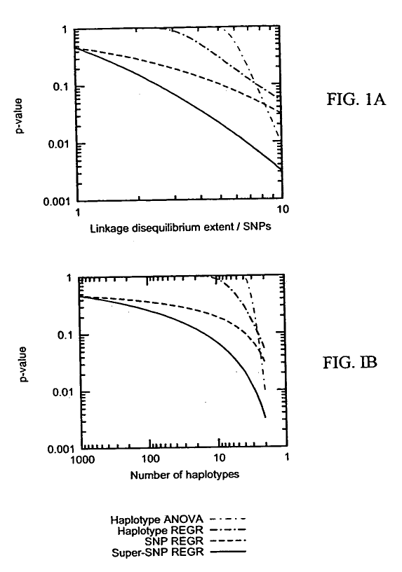

The expected p-values from an association study with a sample size N=150 using these three types of markers, obtained from Eq. 1 for regression tests and Eq. 3 for ANOVA, is displayed in FIG. 1. The abscissas of the top and bottom panels are related by G/Γ=log₂ H. The general behavior for each test is a gain in significance as linkage disequilibrium increases from left to right across the figure. The test providing the smallest p-value uses super-SNPs, followed by the SNP-based test and the haplotype-based regression test. The haplotype-based ANOVA test has less significance than the haplotype-based regression test until there are only 2 or 3 haplotypes, at which point the p-values cross and the ANOVA test is better.

The ratio p-value(SNP)/p-value(super-SNP) reduces to the extent of linkage disequilibrium measured by G/Γ. The test are equally significant when G/Γ=1 and all SNPs are uncorrelated. The super-SNP test is 10-fold more significant when G/Γ=10, complete disequilibrium across the 10 SNPs. If super-SNPs can be identified and the number of super-SNPs is smaller than the number of haplotypes, then the super-SNP test produces a more significant finding than the haplotype test.

If the extent of linkage disequilibrium is difficult to estimate or super-SNPs can not be identified, then it is more reasonable to compare the p-value from a haplotype test based on the observed number of haplotypes to the p-value from a SNP-based test with no linkage disequilibrium, corresponding to G/Γ=1. The ratio of these p-values is
p-value(HAP)/p-value(SNP)=(H/G)^{3/2}exp [N σ_{G}²(1−G/H)/2],
an approximation obtained from the asymptotic expansion of Φ(z) for small z. The haplotype-based test is more significant when the number of haplotypes is smaller than the number of SNPs. Conversely, the SNP-based test is more significant when the number of SNPs is smaller than the number of haplotypes.

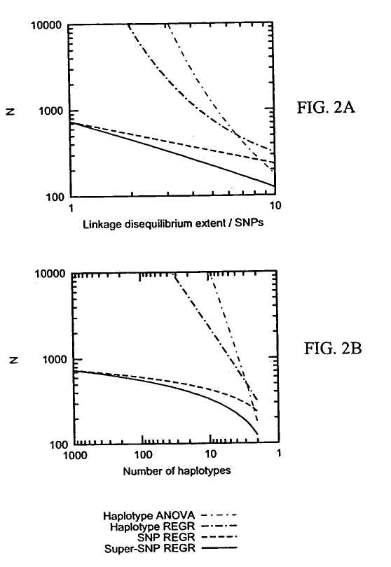

The sample sizes required to achieve a power 1−β=80% to reject the null hypothesis with a Type I error rate α=5% corrected for multiple hypothesis testing are shown in FIG. 2. As in FIG. 1, the top and bottom panels are identical except for a rescaling of the abscissa. The power of each test increases with the linkage disequilibrium from left to right. When the linkage is virtually complete, with only 2 or 3 haplotypes in a population, the haplotype-based ANOVA test is more powerful than the haplotype-based regression test. With slightly less disequilibrium, however, the ANOVA test loses power rapidly.

The most powerful regression test uses super-SNPs, followed by SNP-based and haplotype-based tests. An approximate value for the ratio of the sample sizes required for the SNP-based and super-SNP-based tests is
N_{SNP}/N_{SSNP}=ln(G/α)/ln(Γ/α),
rising from a factor of 1 under weak linkage to a maximum of 1+log_{1/α}(G) under strong linkage. If the extent of linkage disequilibrium is evident and super-SNPs can be identified, the test based on super-SNPs is uniformly more powerful than the haplotype-based test. If linkage disequilibrium is difficult to estimate, then it is reasonable to compare the sample size required by the haplotype-based test for H haplotypes to the sample size required for the SNP-based test assuming the worst case of no disequilibrium. This ratio may be approximated as
N_{HAP}/N_{SNP}=(H/G)ln(H/α)/ln(G/α).
Haplotype-based tests are more efficient than SNP-based tests when there are fewer haplotypes than SNPs and less efficient when there are more haplotypes than SNPs.

Sample size estimates for other values of the fractional variance contributed by the polymorphisms, fixed at 5% in this example, may be readily determined from FIG. 1 because N is inversely proportional to this variance.

Additional embodiments are within the claims.

The invention will be further illustrated in the following non-limiting examples.

### EXAMPLE 1

### Comparison of Association Studies at the Gene Encoding the β₂-Adrenergic Receptor (β₂AR)

This example concerns association studies using the gene encoding the β₂-adrenergic receptor (β₂AR). This G-protein coupled receptor is expressed in airway smooth muscle cells and mast cells and is the target of bronchodilating β-agonists such as isoprenaline, salmeterol, and albuterol used in the treatment of asthma [Goodman and Gilman's The Pharmacological Basis of Therapeutics, Ninth Edition. Goodman L S, Hardman J G, Limberd L E, Molinoff P B, Ruddon R W, Gilman A G (Eds.). McGraw Hill, New York (1996)]. Polymorphisms at codons 16 (arg to gly) and 27 (gln to glu) have been associated at varying levels of significance with response to β-agonist treatment [Tan et al., Lancet. 350: 995-999, 1997; Taylor et al., Thorax. 55: 762-767, 2000; Chong et al., Pharmacogenetics.10:153-162, 2000; Liggett, J. Allergy Clin. Immunol. 105:S487-S492, 2000]. Between the β₂AR transcription start site and the intronless coding region is a 5′-leader cistron which encodes a 19-aa peptide, and polymorphisms in this region have been shown to affect β₂AR expression [McGraw et al., J. Clin. Invest. 102: 1927-1932, 1998]. To understand the relevance of these and other polymorphisms in (β₂AR, Liggett and coworkers undertook an association study focusing on the relationship between SNPs, haplotypes, and response to the bronchodilator albuterol [Drysdale et al., Proc. Natl. Acad. Sci. USA 97: 10483-10488, 2000].

In a scan of chromosomes from 23 Caucasians, 19 African-Americans, 20 Asians, and Hispanic-Latinos, the Liggett study identified a total of 13 polymorphic sites in a region including ˜700 nt of ORF and ˜1100 nt of 5′ UTR, including the 5′-leader cistron. While 12 total haplotypes were identified, only 4 had frequency above 5% in any ethnicity, and only 3 of these occurred at 2% frequency or greater in the Caucasian population. In these 3 haplotypes, 10 of the 13 SNPs were variable. The SNPs and haplotypes were then tested for association with albuterol response, adjusted for sex and baseline severity, in a population of 121 Caucasian patients with moderate asthma. A haplotype association test was performed using ANOVA for the 5 haplotype pairs observed in the treated population, and SNP main effects were tested using ANOVA for SNP genotypes with p-values corrected for multiple hypothesis testing. While the haplotype-based test yielded a significant finding at a p-value of 0.007, none of the SNP-based tests was significant at a p-value of 0.05. The parameters used to analyze these findings are H=3 haplotypes, G=10 of the 13 SNPs which vary in these haplotypes, and C=10 possible pairwise comparisons between the 5 haplotype pairs.

Using Eq. 3, the characteristic haplotype contribution to the phenotypic variance, σ_{H}², may be estimated from the haplotype-based ANOVA to be 0.063. Had haplotype-based regression been performed instead of ANOVA, use of Eq. 1 predicts that a p-value of 0.008 would have been observed. Although the small number of haplotypes suggests strong linkage disequilibrium between SNPs, sequence data presented by Martin and coworkers demonstrates that correlation between SNPs extends no further than one or two SNPs, in accord with their observation that no SNP correlated perfectly with any haplotype. Consequently the weak linkage limit, i.e., no SNP correlation, is used to estimate the expected p-value from a SNP-based regression test. The resulting p-value from Eq. 1, corrected for multiple hypothesis testing, is 0.49, consistent with the reported lack of significance. The Liggett study is therefore consistent with a model of simple additive effects from multiple causative SNPs; there is no indication of unique or non-additive interactions. Although such effects can not be ruled out, it is not likely that this series of experiments, with insufficient power to detect the simple main effect of individual SNPs, would have sufficient power to detect the interaction terms in an ANOVA model. Similarly, although a model including haplotype main effects and haplotype-haplotype interactions would be expected to yield significance for the main effects, it is unlikely that the interaction terms would be significant.

### EXAMPLE 2

### Comparison of SNP-Based and Haplotype-Based Association Studies

This example provides an illustration of the methods of the invention using data presented in a series of simulations designed to assess the power of various association studies. Long & Langley, Genome Res. 9: 720-731, 1999]. Although the details of the simulation model, including the use of haploid rather than diploid genomes for estimates of the power of haplotype-based association studies, are different from the model considered here, the essence of the model is the same: multiple polymorphic markers exist in linkage disequilibrium with each other and with a quantitative trait nucleus. Long and Langley report, based on their simulations, that tests which consider each single marker in turn have power similar to or greater than haplotype-based tests. The same conclusion is reached with the present analytical results, provided that the total number of haplotypes is larger than the total number of SNPs.

Long and Langley also investigate the effects of increasing marker density relative to a parameter 4Nc, a measure of the extent of linkage disequilibrium along a chromosome. Once the marker density is comparable to the inverse of this length, the simulation results suggest that it is more powerful to increase the number of individuals genotyped than to increase the number of markers tested. The present findings are similar, with the extent of linkage disequilibrium expressed as the number of consecutive SNPs correlated between different haplotypes. Furthermore, when the SNP density is so high that SNPs form super-SNPs, it is found that additional SNPs may actually decrease the power of a SNP-based test due to the correction for multiple hypothesis testing.

### EXAMPLE 3

### Comparison of SNP-Based and Haplotype-Based Tests Using Varying Numbers of Causative SNPs

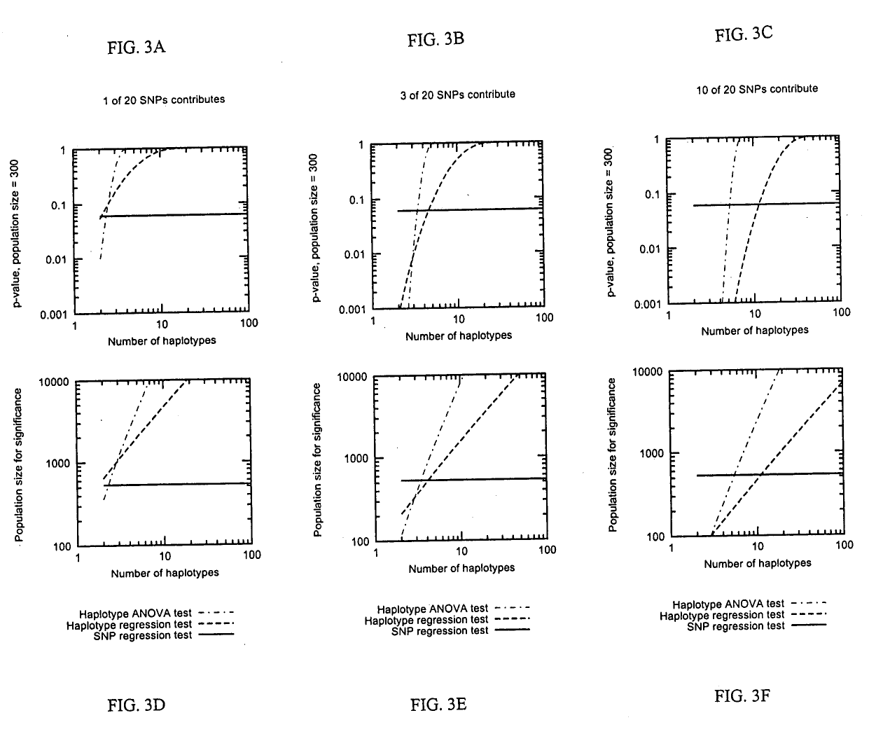

A comparison of SNP-based and haplotype-based tests is presented in FIGS. 3A-3F using a fixed total number of SNPs and a varying number of causative SNPs. The number of total number of SNPs is fixed at 20. The number of causative SNPs is 1 (left panels), 3 (middle panels), or 10 (right panels). The number of haplotypes, H, is varied from 1 to 100 within each panel. The additive variance per SNP is fixed at 0.025. The top series of panels illustrates the expected significance for a fixed population size of 300, and the bottom series illustrates the population size required to attain a p-value of 0.05 (5% false-positive rate including the multiple-testing correction) and a power of 0.8 (20% false-negative rate), for the haplotype-pair ANOVA test (dot-dashed line), the haplotype regression test (dashed line), and the SNP regression test (solid line). Haplotype-based tests and SNP-based tests cross in power when the number of haplotypes is just larger than the number of causative SNPs.

### EXAMPLE 4

### Comparison of SNP-Based and Haplotype-Based Tests Using Fixed Total Additive Variance

A comparison of SNP-based and haplotype-based tests using fixed total additive variance is presented in FIG. 4. The results of the series is similar to FIG. 3, except the total additive variance is fixed at 0.075, implying an additive variance per SNP that varies from 0.075 (1 causative SNP) to 0.0075 (10 causative SNPs). Haplotype-based tests and SNP-based tests cross in power when the number of haplotypes is just larger than the number of causative SNPs.

1. A method of associating a phenotype with the occurrence of a particular set of allelic markers that occur at a plurality of genetic loci in a population of individuals, the method comprising:
a) identifying a phenotype that is expressed by a trait that is quantitatively evaluated on a numeric scale;
b) identifying for each genetic locus of a plurality of genetic loci the form of the allelic marker occurring at a plurality of genetic loci, where said genetic locus is characterized by having at least two allelic forms of a marker and wherein the phenotype is expressed by a trait that is quantitatively evaluated on a numeric scale;
c) identifying a set of said allelic markers present in the nucleic acid of each individual of the population;
d) obtaining the numeric value corresponding to the phenotypic trait for each individual of the population; and
e) obtaining a p-value based on a particular set of markers and the numeric value, wherein the p-value provides the probability that the association of the phenotype with the particular set is due to a random association, whereby obtaining a p-value less than a predetermined limit establishes the association of said phenotype with occurrence of a particular set of a the particular set of allelic markers that occur at a the plurality of genetic loci in a the population of individuals.

2. (canceled)

3. The method of claim 1, wherein the number of individuals is 5,000 or fewer.

4. (canceled)

5. The method of claim 1, wherein the number of individuals is 500 or fewer.

6. (canceled)

7. The method of claim 1, wherein at least one allelic marker is a single nucleotide polymorphism (SNP).

8. The method of claim 1, wherein a genetic locus is characterized by having two allelic forms of the marker.

9. The method of claim 1, wherein at least two genetic loci are in linkage disequilibrium with respect to each other.

10. The method of claim 1, wherein a particular set of allelic markers comprise a haplotype.

11. The method of claim 1, wherein at least two genetic loci comprise a set of super-SNPs.

12. The method of claim 1, wherein the p-value is obtained using a regression analysis or an analysis of variance (“ANOVA”).

13. (canceled)

14. The method of claim 1, wherein the p-value is less than 0.1.

15. (canceled)

16. The method of claim 1, wherein the p-value is less than 0.01.

17. A method of estimating the number of individual samples required to establish the association of a phenotype with occurrence of a particular set of allelic markers that occur at a plurality of genetic loci in a population of individuals, wherein each genetic locus is characterized by having at least two allelic forms of a marker and as being the locus of a set of single nucleotide polymorphisms (SNPs), and wherein the phenotype is expressed by a trait that is quantitatively evaluated on a numeric scale, the method comprising the steps of:
a) determining the number of SNPs to be evaluated;
b) combining consecutive SNPs that are in linkage disequilibrium into super-SNPs;
c) determining the number of haplotypes; and
d) determining the estimated number of samples required.

18. The method of claim 17, wherein the number of SNPs plus the number of super-SNPs is smaller than the number of haplotypes, and wherein the estimating uses the formula provided on the last line of Table 1 in column 2 or column 3.

19. The method of claim 17, wherein the number of SNPs plus the number of super-SNPs is greater than the number of haplotypes, and wherein the estimating uses the formula provided on the last line of Table 1 in column 4.

20. The method of claim 17, wherein the number of haplotypes is 2 or 3, and wherein the estimating uses the formula provided on the last line of Table 1 in column 4 or column 5.

21. The method of claim 17, wherein the number of haplotypes is 4 or more, and wherein the estimating uses the formula provided on the last line of Table 1 in column 5.

22. A method for determining whether a genetic region is associated with a disease, the method comprising:
(a) providing a plurality of single-nucleotide polymorphisms and a plurality of haplotypes for one or more regions of a chromosome;
(b) identifying the number of said single-nucleotide polymorphisms in linkage disequilibrium with each other on said chromosomal regions;
(c) comparing the number of said single-nucleotide polymorphisms in linkage disequilibrium to the number of said haplotypes in said chromosomal regions; and
(d) selecting a correlation test, wherein a single-nucleotide-based correlation test is selected if the number of single-nucleotide polymorphisms in linkage disequilibrium is smaller than the number of haplotypes and a number of haplotype-based correlation test is selected if the number of single-nucleotide polymorphisms in linkage disequilibrium is greater than the number of haplotypes, thereby determining whether said genetic region is associated with a disease.

23. The method of claim 22, wherein the haplotype-based correlation test is a regression test or an analysis of variance (“ANOVA”).

24. (canceled)

25. A method for identifying a genetic region associated with responsiveness to an agent, the method comprising:
(a) providing a plurality of single-nucleotide polymorphisms and a plurality of haplotypes for one or more regions of a chromosome;
(b) identifying the number of single-nucleotide polymorphisms of said plurality in at least weak linkage disequilibrium with each other on said chromosomal regions;
(c) comparing the number of single-nucleotide polymorphisms in linkage disequilibrium to the number of haplotypes in said chromosomal regions; and
(d) selecting a correlation test, wherein a single nucleotide-based correlation test is selected if the number of single-nucleotide polymorphisms in linkage disequilibrium is smaller than the number of haplotypes, thereby identifying a genetic region associated with responsiveness to an agent.

26. The method of claim 25, wherein the haplotype-based correlation test is a regression test or an analysis of variance (“ANOVA”).

27. (canceled)
